(19) United States
(12) Patent Application Publication
(10) Pub. No.: US 2021/0353821 A1
(43) Pub. Date: Nov. 18, 2021

(54) Filament having cellulose fibers with non resorbable features less radio pacifier

(71) Applicant: ROBERT C JOYCE, Toledo, OH (US)

(72) Inventor: ROBERT C JOYCE, Toledo, OH (US)

(21) Appl. No.: 17/316,540

(22) Filed: May 10, 2021

Related U.S. Application Data
(60) Provisional application No. 63/101,802, filed on May 18, 2020.

(51) Int. Cl. A61L 17/00; D06M 15/59; A61L 17/04; A61L 17/10; A61L 15/54; D06M 15/705; D06M 15/227

(52) U.S. Cl. CPC A61L 17/005; D06M 15/59; A61L 17/04; A61L 17/105; D06M 2101/06; D06M 15/705; D06M 15/227; A61L 2300/406; A61L 15/54

(57) ABSTRACT

The filament and suture products disclosed are to be implanted in the body, having non re sorbable ingredients with absorbent qualities to disperse an antibiotic and strong enough to hold tissue securely but flexible enough to be printed or knotted. The products are biocompatible and consist of two dissimilar polymers having unmelted cellulose fiber. These dissimilar polymers and the unmelted fibers are densified by compression and the removal of moisture having fiber orientation and alignment, showing low levels of radiopacity. These products will have radiopacity in household units (HU) ranging from −200 to 200 HU and produce meshes, bone grafts, scaffolds or wound care products where bone bridging can be observed.

### CROSS REFERENCES

Application claims priority of U.S. Provisional Patent Application Ser. No. 63/101,802 filed May 18, 2020, this prior application are incorporated herein by reference.

### FIELD OF INVENTION

Surgical removal procedures for permanent non re sorbable implants like sutures are compromised due to hydrolysis and degrade over a 12-14 week period and have no visibility in the body. Unless traditional radio pacifiers are utilized in the sutures there are unknowns as to implant movement and body acceptance. By altering monomeric components and relative compositions, polymers can be tuned to provide specific mass and strength loss profiles that match a required function, such as wound healing or surgical aids. The inventive non re sorbable, biocompatible filament and suture strands identified in this specification have radiopacity without radio pacifiers like barium sulfate or bismuth, a much improved resistance to hydrolysis and hydrophilicity for coatings to disperse antibiotics. Furthermore, these filaments or other constructs such as sutures or threads will have radiopacity to identify bone bridging in meshes, bone grafts or scaffolds. These filaments, sutures, threads will have oriented cellulose fiber with two dissimilar thermoplastic polymers, wherein the cellulose fibers are densified without having any visible agglomeration or clumping at a range of 2× to 4× magnification with a 4 mm field of view. These filaments are produced with a outside diameter (O.D.) at less than 3 mm and the sutures having a outer diameter (O.D.) no larger than 0.1 mm. This inventive filament can be processed through various extrusion methods including a ram type, die drawn method to produce a suture or spinning to produce a thread.

It is known sutures are classified as either absorbable or non-absorbable depending on whether the body will naturally degrade and absorb the suture material over time. Most sutures are synthetic including absorbable suture materials: the original catgut as well as one or more of five cyclic monomers: glycolide, l-lactide, p-dioxanone, trimethylene carbonate and ε-caprolactone. These absorbed bodily fluids and could be foci of infection. The non-absorb able sutures are radiolucent polymers including nylon, polyester, PVDF and polypropylene. The non re sorbable suture types like nylon are hydrophilic, will absorb moisture, can degrade in less than 12 weeks, have low levels of surface energy below 50 dynes/cm.

The sutures depending on location of injury and type of wound will require a specific suture size and strength that have re sorbable or non re sorbable features. Commonly referred to as a re sorbable suture, or having it dissolve by the body, i.e. PLLA, PLA, the suture location, size and composition, it may dissolve and degrade at different rates. These characteristics of re sorbable sutures or threads made to make products like meshes, scaffolds indeed comprise healing of the tendon or wound.

The inventive filament and suture implant products are a cellulose fiber thermoplastic composition comprising two synthetic dissimilar polymers, having different polarity with cellulose fiber, an organic polymer. The non polar polymer is polyolefin such as polypropylene, polyethylene and the polar polymer can be a polyamide occurring both natural or synthetic. The cellulose fiber has at least 95% purity and is blended with polar and non polar thermoplastics and compatibalizer. The compatibalizer can be reactive or non reactive. The composition can include additives, i.e. proteins, zinc, hydroxyapatite, magnesium, elastomer, stearate, collagen, antibiotic.

Unlike the traditional surgical implants, these inventive filament and suture products can be coated, whereby they have a dyne level range of 50 dynes/cm to 100 dynes/cm. The dyne level or surface energy of the products will promote increased coating functionality or compatibility without requiring special processing. The extruded filaments, sutures, threads referred to as “the products” will be coated in line with a liquid such as a collagen dispersing the antibiotic to produce a superior wound care mesh, bone graft or any scaffold.

### THE BACKGROUND OF INVENTION

The textile market for absorbable's is currently dominated by sutures and mesh material, although there is growing interest in absorbable polymers for new uses, such as tissue scaffolding and temporary or semi-permanent complete device platforms. The current design limitations on such devices generally stem from materials able to satisfy more diverse combinations of mass and strength loss profile requirements with specific mechanical or physical conditions.

Non-absorbable sutures are made of special silk or the synthetics polypropylene, polyester or nylon. Stainless steel wires are commonly used in orthopedic surgery and for sternal closure in cardiac surgery. These may or may not have coatings to enhance their performance characteristics. Non-absorbable sutures are used either on skin wound closure, where the sutures can be removed after a few weeks, or in stressful internal environments where absorbable sutures will not suffice. Examples include the heart (with its constant pressure and movement) or the bladder (with adverse chemical condition.

A coating has been necessary to identify filaments, sutures, thread or products such as meshes, bone grafts, implants a previous needs to improve patient safety. A reference has been identified where a suture having radiopaque coatings is highly preferred by Ken Morabito author of Radiopaque medical devices improves patient safety published in October 2013. This study represents many un retrieved medical device occurrences that may be preventable by using post procedure diagnostics to rule out any retained medical devices. Radiopaque additives (radio pacifiers) are used in medical devices to increase their visibility using x-ray or other fluoroscopy. This serves to improve post procedure awareness of retained devices or fragments as well as to provide markers for better placement and guidance of medical devices.

The filaments used for medical applications must be hypoallergenic and avoid the “wick effect” that would allow fluids or contain microbial additives or healing accelerators and thus reducing chances for infection to penetrate the body along the suture tract. The inventive filament and products construction has polyamide which has demonstrated anti bacterial properties, cellulose fibers that are non re sorbable with low levels hydrophilicity and considered absorbent. The inventive filament also has polyolefin which is hydrophobic with no adverse effects to the body. The overall construct of the printed filaments has shown cell and tissue adhesion, often referred to osteo conductive properties. Furthermore, the cellulose fiber will assist in holding tissue securely and with the filament flexible enough to be printed or knotted. The hydrophilic properties of the filaments, sutures can be coated with antibiotics and or anti microbial additives for accelerating healing and reducing infection. The disclosed filament and suture implant products will replace a existing radiolucent polymers such as nylon and polypropylene, to be woven into a mesh or braided for applications such as wound care.

### DESCRIPTION OF RELATED ART

Suture thread is made from numerous materials. Sutures come in very specific sizes and may be either absorbable (naturally biodegradable in the body) or non-absorbable. The non-absorbable sutures often cause less scarring because they provoke less immune response, and thus are used where cosmetic outcome is important. They may be removed after a certain time, or left permanently. Nylon (eg ethilon) is a synthetic monofilament material widely used for skin suture.The Ethilon® Nylon suture is a non-absorbable, monofilament, synthetic suture that is commonly used for cutaneous wound closure. It is popular among surgeons because of its high tensile strength, low tissue reactivity and good elasticity. Polypropylene (prolene) is often preferred to nylon sutures as it is thought to be slightly more inert. The polypropylene suture is safe in most situations, but care must be taken to avoid instrumentation trauma and kinking stresses at knots, which probably explain most of the reported cases of polypropylene failure. In addition, polypropylene probably should not be used in graft-to-graft anastomoses in which the continual sawing stresses of two rigid structures.

Some sutures are intended to be permanent, and others in specialized cases may be kept in place for an extended period of many weeks, as a rule sutures are a short term device to allow healing of a trauma or wound. Different parts of the body heal at different speeds. Common time to remove stitches will vary: facial wounds 3-5 days; scalp wound 7-10 days; limbs 10-14 days; joints 14 days; trunk of the body 7-10 days. Removal of sutures is traditionally achieved by using forceps to hold the suture thread steady and pointed scalpel blades or scissors to cut. For practical reasons the two instruments (forceps and scissors) are available in a sterile kit. In certain countries (e.g. US), these kits are available in sterile disposable trays because of the high cost of cleaning and re-sterilization.

Radiopaque suture patent WO2015179167A1 Jackson et al. A polyamide suture includes an elongate core formed of multiple twisted and beat set filaments formed of a first polyamide material and a sheath forced of a second polyamide material surrounding the core along its length, the second polyamide material having dispersed therein non-absorbable radiopaque nanoparticles comprising 15-25% by weight, of the second polyamide material. The melting point of the first polyamide material is at least 30° C. greater than the melting point of the second polyamide material. The core may also include or be formed of previously extruded bundles of first polyamide filaments overcoated with a second polyamide sheath. The suture is made by co extruding the core arid a molten organic material formed of the second polyamide material and dispersed radiopaque nanoparticles. These nanoparticles used for radiopacity were tantalum by Jackson et al. because of the radiolucency of the plastic. Tantulum is a radio pacifier as well as, tungsten, bismuth and barium sulfate which is frequently included in this plastic compound. Other medical devices used a combination of Nylon, Propylene materials use as radiopaque markers in Baker et al. include a fiber can comprise a polymer, a radiopaque material, and a dye/colorant. Non-limiting examples of a polymer are polypropylene and nylon. In certain aspects, the only polymer in the radiopaque tissue marker is polypropylene. Again common radio pacifiers are cited in Non-limiting examples of a radiopaque material are barium sulfate, bismuth, tantalum, and iodine. In certain aspects, barium sulfate has a weight percentage greater than 40% in the radiopaque tissue marker. In certain aspects, the radiopaque material is not a radiopaque heavy metal. Non-limiting examples of a dye are a copper phthalocyanine blue, such as C.I. Pigment Blue

These non-absorbable sutures often cause less scarring because they provoke less immune response, and thus are used where cosmetic outcome is important. They may be removed after a certain time, or left permanently. The polymers used to produce catheters and other devices that are inserted into the body for diagnostic or interventional procedures are commonly filled with substances opaque to x-rays, thereby rendering the devices visible under fluoroscopy or x-ray imaging. These fillers, or radiopacifiers—typically dense metal powders—affect the energy attenuation of photons in an x-ray beam as it passes through matter, reducing the intensity of the photons by absorbing or deflecting them. Because these materials exhibit a higher attenuation coefficient than soft tissue or bone, they appear lighter on a fluoroscope or x-ray film. This visibility provides the contrast needed to accurately position the device in the affected area. Image contrast and sharpness can be varied by the type and amount of radio pacifier used, and can be tailored to the specific application of the device. For example, a device designed for use near the surface of the skin requires less radiopaque filler to achieve the required level of attenuation compared with one used inside the coronary vasculature. Device design is also a factor: a higher loading of radiopaque material, for instance, is needed for thin-wall catheter tubing than for products with thicker walls. Generally, compounds should contain only the amount of additives absolutely required for the application, since overloading can result in the loss of the polymer's mechanical properties. Blending together several radiopaque materials can produce better results than using only one type in a formulation. Barium sulfate has a specific gravity of 4.5, and generally used at loadings of 20 to 40% by weight. While a 20% barium sulfate compound is typical for general-purpose medical device applications, some practitioners prefer a higher degree of radiopacity than can be provided by that loading. With striped tubing, for example, a 40% compound is standard.

Considerably more expensive than barium at $20 to $30/lb (depending on the chemical salt selected), bismuth compounds are also twice as dense. Bismuth trioxide (Bi2O3), which is yellow in color, has a specific gravity of 8.9; bismuth subcarbonate (Bi2O2CO3) has a specific gravity of 8.0; and bismuth oxychloride (BiOCl) has a specific gravity of 7.7. Because of the density, a 40% bismuth compound contains only about half the volume ratio as a 40% barium sulfate compound. Since bismuth produces a brighter, sharper, higher-contrast image on an x-ray film or fluoroscope than does barium, it is commonly used whenever a high level of radiopacity is required. Tungsten, a fine metal powder with a specific gravity of 19.35, tungsten (W) is more than twice as dense as bismuth and can provide a high attenuation coefficient at a cost of approximately $20/lb. A loading of 60% tungsten has approximately the same volume ratio as a 40% bismuth compound. Devices can be made highly radiopaque with relatively low loadings of tungsten, enabling good mechanical properties to be maintained. Because of its density, tungsten is typically selected as a filler for very-thin-walled devices. Jianmin Li has patented Surgical meshes with radiopaque coatings U.S. Pat. No. 9,427,297 where implantable medical articles are provided, which comprise a surgical mesh that is at least partially covered with a coating that comprises a radiopaque material such as a metal or a metallic compound. The radiopaque material is present in the coating in an amount such that the coated portions of the mesh are visible using radiographic imaging techniques. Other aspects of the invention pertain to methods of making and using such medical articles.

Newer x-ray machines generally operate at higher energy levels than older ones—typically at 80 to 125 kVp as compared with 60 to 80 kVp for older machines. Higher energy radiation increases the transmission of photons and can require higher levels of radiopacity to provide the desired attenuation. Therefore, devices produced with barium sulfate compounds might not appear as bright on newer machines, for which bismuth compounds would be a better choice of radiopaque filler.

The radio pacifiers are not all compatible with thermoplastics, difficult to disperse, can compromise the strength of the filament with little or no compressibility and the cost of the ingredients are problematic for commercialization. The inventive filament and suture strands, constructed with a cellulose fiber thermoplastic composition without radio pacifiers, can be densified to a specific gravity no less than 1. The filaments are constructed of synthetic polymers comprising unmelted cellulose fibers which are compress able at pressures beyond 175 psi to assist in producing radiopacity for improved implant identification and patient outcomes.

### BRIEF SUMMARY OF THE PRESENT INVENTION

The disclosed filament strand and suture strand for an implant are comprised of a densified cellulose fiber with two dissimilar thermoplastic polymers and referred to as a cellulose fiber thermoplastic composition. The densified cellulose fiber thermoplastic filament, suture or thread will have low levels of radiopacity with non re sorbable ingredients. These products are biocompatible for implanting in the body, strong enough to hold tissue securely but flexible enough to be printed or knotted and have absorbent qualities for tissue or cell to have growth on or in a bone graft, scaffold or mesh. This composition can also be coated with an antibiotic for accelerating the healing process. The cellulose fiber thermoplastic composition can be spun into threads for producing meshes or wound care products. Furthermore, an extrusion method of the cellulose fiber thermoplastic may produce filaments for 3D printed bone grafts, scaffolds or meshes.

The non re sorbable sutures like nylon has tensile strength that ensures wound security, however, the disadvantage of nylon is the challenge in tying a secure knot and the degradation of the nylon resin. Furthermore, monofilaments have greater memory (the tendency to return to their packaged shape) than braided sutures, they tend to unravel if not tied correctly. By combining a polyolefin with the polyamide, a Nylon 6, or Nylon 66 less degradation is possible and by adding the cellulose fiber additional reduction in degradation occurs. The suture strand or filament strand produced with a polyamide and polyolefin and compatibalizer with cellulose fiber is non re sorbable with an ability to deliver antibiotics and print micropore structures. Furthermore, the glass transition temperatures of the cellulose fibers exceeds 100 C to assist in forming a very tiny micropore with the polar and non polymer synthetic polymers. Each synthetic polymer described in this specification has and individual glass transition temperature. The polyamide glass transition temperature is 60 C, while polypropylene is −10 C. The cellulose fiber thermoplastic composition glass transition temperature can be in a range of 50 to 80 C in producing a suture and or filament strand to print a bone graft, scaffold or mesh having a 60 micron pore size and higher. A die drawn or ram extrusion method to produce the specified filament having less than 1 mm to 3 mm size is preferred. Furthermore, a spinning process can produce threads for weaving a small porous mesh with less than 50 micron size pores. The cellulose fiber in the thermoplastic matrix used in spinning will have unmelted fibers, where the fibers are not exposed to temperatures above 260 C and the die pressure exceeds 175 psi.

By processing the composition above 260 C, the cellulose fiber will be melt blended into the thermoplastic composition, adversely affecting radiopacity and strength of the cellulose fiber thermoplastic composition. The cellulose fibers in the thermoplastic polymers will have reduced performance, a brown color and considered decomposed. These cellulose fibers with the two dissimilar thermoplastic polymers will require moisture removal of 6-7%. The controlled moisture removal will assist in dispersion of the cellulose fibers in the thermoplastic compound, producing a plasticizer and reducing brittleness in the composition. It is significant the cellulose fibers are oriented and not clumped or agglomerated and the composition is densified in producing the filament or suture strands to implant in the body. In some instances where an increase of radiopacity is required for the strands, cellulose fiber in the composition can be adjusted to exceed 10% by volume in the polyamide and polyolefin composition. Another preferred filament strand or suture strand has a cellulose fiber content of 14% with two dissimilar thermoplastic polymers, having moisture removed with die pressure greater than 275 psi and a specific gravity of 1.15.

The polyamide utilized in the inventive filaments, sutures and threads is a macromolecule with repeating units linked by amide bonds. Any of these polyamides occur both naturally and artificially. An example of naturally occurring polyamides are proteins, such as wool and silk. Artificially made polyamides can be made through step-growth polymerization or solid-phase synthesis yielding materials such as nylons, aramids, and sodium poly(aspartate). By using a aromatic polyamide the ability to sterilize is enhanced for the implantable suture. This polymer has less absorption of fluids and can be more receptive to acting as an amorphous semi crystalline polymer. The polyamides are any range of polymers containing amide (or peptide) repeat units; examples include proteins and nylon. Nylon is a generic designation for a family of synthetic polymers, based on aliphatic or semi-aromatic polyamides. The medical thread made with polyamide and polyolefin with cellulose fiber can be processed with an amorphous nylon and or amorphous polyolefin.

The cellulose fiber thermoplastic composition requires multiple processing with vents to achieve optimum performance for an implants such as sutures, meshes, scaffolds or bone grafts. An initial process must include melt blending, shearing of the preferred polar polyamide and non polar polyolefin with a compatibilizer in a high shear twin screw extruder. The initial extrusion of these polar and non polar synthetic polymers will have processing temp at throat of the twin screw compounder between 260-280 c with a vent for moisture removal. Additional melt blending with additives such as elastomer, minerals can be blended to produce the compounded pellets. These compounded pellets will then be processed in a secondary process or separate extrusion process where the cellulose fiber is added to the thermoplastic composition at temperatures range of 198-213 C. Additional additives like lubricants and adhesives may be compounded with the cellulose fiber thermoplastic composition. The cellulose fiber in thermoplastic compound will have unmelted fibers in a pellet form for extruding filaments, sutures or threads. These pellets will have a moisture levels less than 2% and will be dried to a preferred moisture range of 0.04%-0.15% before compressing and or processing the filaments, sutures or threads having a continuous strands. The biocompatible filaments and suture strands will be extruded below 260 C and will have water or moisture present as a plasticizer with unmelted cellulose fibers. The cellulose fiber will be compressed and densified and absence agglomeration, as visibly seen in a 2×, 4× magnification with a field of view at 4 mm. If the fibers are exposed to excessive shear and or prolonged heat exposure, the fibers can melt to compromise fiber performance and decompose. In some filaments, sutures and threads, there is a mix of unmelted and melted cellulose fibers.

The compounded polyamide and polyolefin with cellulose fiber produces a filament, sutures and threads with radiopacity ranging from −200 to 200 HU. The thread from the compound will range from 0.02 mm O.D. to 0.002 mm for a woven textile which are ‘twisted’ and or ‘plied’. Since filaments of this small diameter are extremely fragile and are difficult to handle, fibers are supplied as untwisted or twisted bundles of filaments. The filament strand from the compound for 3D printing medical devices and or implants are of a size range 1 mm to 3 mm and has been printed with a nozzle size range of 0.2 mm O.D to 0.8 mm. The suture strand from the compound is produced having a size range of 0.1 mm to 0.9 mm O.D.

### DETAILED DESCRIPTION OF INVENTION

The inventive non re sorbable filaments and suture strands implantable in the body are constructed of a cellulose fiber thermoplastic composition that comprises unmelted cellulose fiber with two dissimilar thermoplastic polymers. The products are biocompatible for implanting in the body and exhibit low levels of radiopacity. The suture strands can be a monofilaments or braided to having an O.D. size range of 0.1 mm to 0.9 mm with sizes #5 (heavy braided suture for orthopedics) to #11-0 (fine monofilament suture for ophthalmics). The cellulose fibre thermoplastic braided suture can comprise two or more strands twisted together. The non braided sutures, also known as monofilament, can be made of single strand comprising cellulose fiber with two dissimilar thermoplastic polymers and compatibalizer. These products will lack the traditional or common radio pacifiers like barium sulfate, bismuth or metals i.e. tungsten and will have a surface energy greater than 50 dynes/cm and below 100 dynes/cm. In order to achieve a desirable product strength and flexibility, moisture removal is controlled in the composition before processing the filaments and suture stand and products.

The cellulose fiber in the thermoplastic composition is required to have less than <120 micron particle size with an aspect ratio ranging from 12:1 to 3:1 and a moisture content not to exceed 10%. The cellulose fiber is preferably extracted from wood pulp but can also be a cotton derivative. The cellulose fiber that is a organic polymer and isn't degradable by hydrolysis in the human body. The cellulose fiber is similar to collagen fiber and when compressed with the thermoplastic polymers, fibers are densified, moisture is removed and the fibers are oriented and aligned by the extrusion or a spinning process. A microcrystalline cellulose with a particle size less than 20 micron can be blended independently or with the cellulose fiber in the thermoplastic polymer compound. In the thermoplastic compound, a polar polyamide comprises a semi crystalline and or amorphous Nylon 6 or 6/6, 11,12, 6i6t. The non polar polyolefin can be a homo or co polymer polypropylene and or polyethylene. The compatibalizer can be identified as a processing aid comprising fatty acids, salts or esters and amides with a fluoropolymer for melt blending the polyamide and polyolefin.

The polyamide and polyolefin with compatibalizer are compounded to generate a pelletized thermoplastic composition. Then, the cellulose fibers are blended, heated with the thermoplastic composition and densified to form the pelletized cellulose fiber thermoplastic composition. The pelletized composition is preferred to be desiccant dried to 0.05% and not completely dry before extruding and compressed above 175 psi for densification to produce filaments, sutures or threads. The extruded composition to produce the filaments or suture products will be a semi solid state, wherein the cellulose fiber has unmelted fibers oriented through a die with at least a 10:1 LAN length ratio and a polymer drawdown at 15-60%. This die drawing of the thermoplastic cellulose fiber alloy composition can be independent of the extruder or through a die attached to the extruder. If the die drawing of the composition is performed independently, a heat method utilizing a heat induction mechanism is desired. The heat induction mechanism will be able to bring the solid state profile to desired melt quickly. Once the cellulose fiber thermoplastic composition has fiber orientation and alignment through a die drawing, there is an increase in tensile modulus of the filament and suture strand. The tensile modulus increase is 15-30% of the cellulose fiber thermoplastic composition's molded and tested value of 1615 MPa using the ISO 527 method. The tensile strength of a suture is measured pounds of tension that the strand will withstand before it breaks when knotted. This innovative high strength filament strand can be seen under X Ray and CT scan not utilizing any common radio pacifier as identified.

The cellulose fiber thermoplastic composition requires multiple processing to achieve optimum performance for a filament, suture or thread or mesh. The polyamide and polyolefin is processed with a compatibilizer by a single or twin screw extrusion method or a high intensity mixer. The melt blending and shearing of the dissimilar polymers will produce a thermoplastic pellet. A secondary extrusion process is preferred, where the cellulose fiber is compounded, blended with the pelletized composition comprising of a polyamide and polyolefin and compatibalizer. The cellulose fiber is added to the polyamide and polyolefin composition in line or side fed downstream at temperatures not greater than the melting temperature of the cellulose fiber at 250-260 C. The preferred temperature or optimized processing temperature of the cellulose fibers with the thermoplastic pelletized composition is a range of 190-210 C. The cellulose fiber is preferred to have less than 8% moisture content when side fed to disperse with the thermoplastics composition. The cellulose fiber can be pre dried before extruding for improved dispersion in the composition. A vacuum and vent is required to remove 6-7% moisture in the cellulose fiber thermoplastic composition.

Example: The extruded semi crystalline polymers with an amorphous polymer is pelletized. The composition comprises 4 pellets, having 85% polyamide, a Nylon 6 with Nylon 6i6t and 10% polyolefin and 5% compatibalizer. These pellets are extruded to produce one pelletized thermoplastic compound. The thermoplastic pellet is then processed with additives at the throat of the secondary extruder, preferably a twin screw, having shear elements at the throat and very low shear elements where the cellulose fibers are fed into the side of the extruder barrel. The cellulose fibers are processed with the thermoplastic composition at 400 F or 204 C at zone 5 or 6. The operator controls the temperature zones in either a reverse profile −270 C at the throat or zone 1 and 193-210 C near breaker plate. Or, a bell shaped profile, which are include zone 1 at 260 C, 254 C in zone 2, 254 C, in zone 3, 237 C zone 4, 215 C, zone 5 side feeder, 204 C F, zone 6, 204 C, zone 7, 198 C and zone 8 198 C.

The secondary extrusion process melt blends and removes 6-7% moisture from the composition and produces a pelletized cellulose fiber thermoplastic composition. The pelletized composition will have less than 2% moisture and unmelted cellulose fibers for producing the filament, suture, thread.

This pelletized thermoplastic cellulose fiber composition would then be desiccant dried to a preferred pellet moisture content of 0.05 to 0.18% before processing into a filament strand. This filament strand, as well as, suture strands will be continuous until cut to predetermined lengths. The cellulose fiber thermoplastic filament strand or suture strand would be extruded at temperatures below 260 C so that the cellulose fiber would have unmelted fibers. This heat profile for processing filament would have a temperature range from 204 C to 255 C and would require a high compression screw and or profile die with a 10:1 LAN. The preferred die pressure exceeds 230 psi for producing an oriented fiber filament with a 0.70 mm O.D. By compressing the cellulose fiber with the thermoplastic composition, moisture is removed and the density was increased, having a 1.06 specific gravity. A test plaque was produced showing no agglomeration or clumping in a 2×, 4× magnification range with a 4 mm field of view.

Most preferably, a cellulose fiber particle size to produce the filament strand in example 1 comprises a 20 to 30 micron range and has 99.5% purity with 0.5% ash content. Any filament, suture, thread products can have cellulose fiber particle sizes to include 30 and 50 micron with aspect ratio ranging from 3:1 to 6:1 and may include aspect ratios of 10:1 and 12:1. Threads can comprise microcrystalline cellulose having a particle size of 1 to 13 micron with aspect ratios of 3:1 and 2:1.

1. A non re sorbable suture strand having densified cellulose fibers with two dissimilar thermoplastic polymers comprising of polyamide and polyolefin and compatibalizer has moisture and is compressed at pressures greater than 175 psi, and, wherein the cellulose fibers have unmelted fibers with alignment and have radiopacity in the body.

2. The non re sorbable suture strand in claim 1, wherein the non re sorbable suture strand having a size range of 0.1 mm to 0.9 mm O.D.

3. The non re sorbable suture strand in claim 1, wherein the non re sorbable suture strand before compression has a moisture content range of 0.04 to 0.15%.

4. The non re sorbable suture strand in claim 1, wherein the non re sorbable suture strand is biocompatible with radiopacity range of −200 HU to 200 HU

5. A non re sorbable filament strand with a cellulose fiber glass transition temperature above 100 and two dissimilar polymers comprising of polyamide and polyolefin and compatibalizer having a specific gravity no less than 1, wherein the cellulose fiber has unmelted fibers with orientation and the composition requires 6% moisture removal and is densified for printing pores above 60 micron.

6. A non re sorbable filament strand in claim 4, wherein the non re sorbable filament strand is biocompatible with a dyne level range of 50 dynes/cm to 100 dynes/cm.

7. A non re sorbable filament, suture, thread implant with cellulose fibers having radiopacity with a range of −200 to 200 HU, and wherein the cellulose fiber is densified with two dissimilar polymers comprising of polyamide and polyolefin and compatibalizer and the fibers are unmelted and melted with an absence of agglomeration at 2× to 4× magnification at 4 mm field of view.

8. A non re sorbable filament, suture, thread implant in claim 6, wherein the non re sorbable filament, suture, thread implant has cellulose fiber particle sizes ranging from 30 to 50 micron with purity above 98%
